(19) United States
(12) Patent Application Publication
(10) Pub. No.: US 2002/0173823 A1
(43) Pub. Date: Nov. 21, 2002

(54) Sense organs synthesizer

(76) Inventor: William James English, Marshall, TX (US)

Correspondence Address: WILLIAM JAMES ENGLISH, P.O. BOX 11, MARSHALL, TX, 75671, US

(21) Appl. No.: 10/011,798

(22) Filed: Apr. 1, 2002

Related U.S. Application Data
(60) Provisional application No. 60/254,469, filed on Dec. 11, 2000.

(51) Int. Cl. A61N 1/00

(52) U.S. Cl. 607/1; 607/118; 128/899

(57) ABSTRACT

The SENSE ORGANS SYNTHESIZER, for BLIND, DEAF, and BALANCE ORGANS IMPAIRED, synthesizes three types of sense organs (EYES, EARS, or BALANCE ORGANS). The senses are ‘perceived’, as if synthetic organs are real. My invention does NOT perform this; it is a feat of the SUBCONSCIOUSNESS. What is new about my invention is simple; I presume upon the following AUTONOMIC FUNCTIONS of subconsciousness: 1) (It) MUST analyze any and all organized/patterned subliminal signals for the meanings, 2) then it presents the meanings (not the original signals) to the consciousness. 3) The subconscious “has no choice” in these matters, is totally unprejudiced, and cannot even refuse these functions. A camera, microphone, or balance sensors, are input to a programmed microprocessor, that digitizes video, audio, or balance signals, such that they can be comprehended by the subconscious. Delivery is through linear arrayed electrodes' signals, to a few hundred TOUCH NERVE ZONES. Stimulation is by Transcutaneous Electric Nerve Stimulus (TENS).

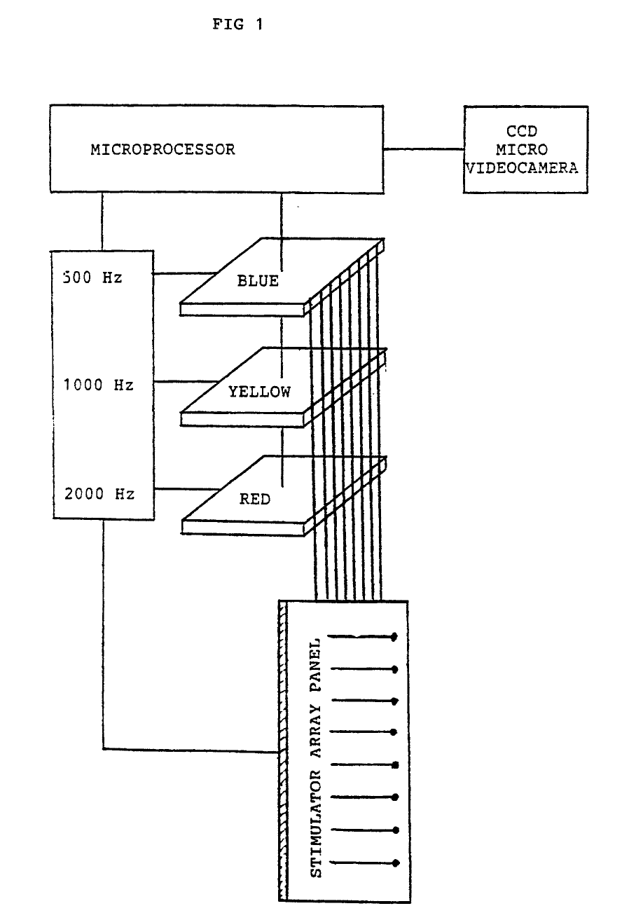

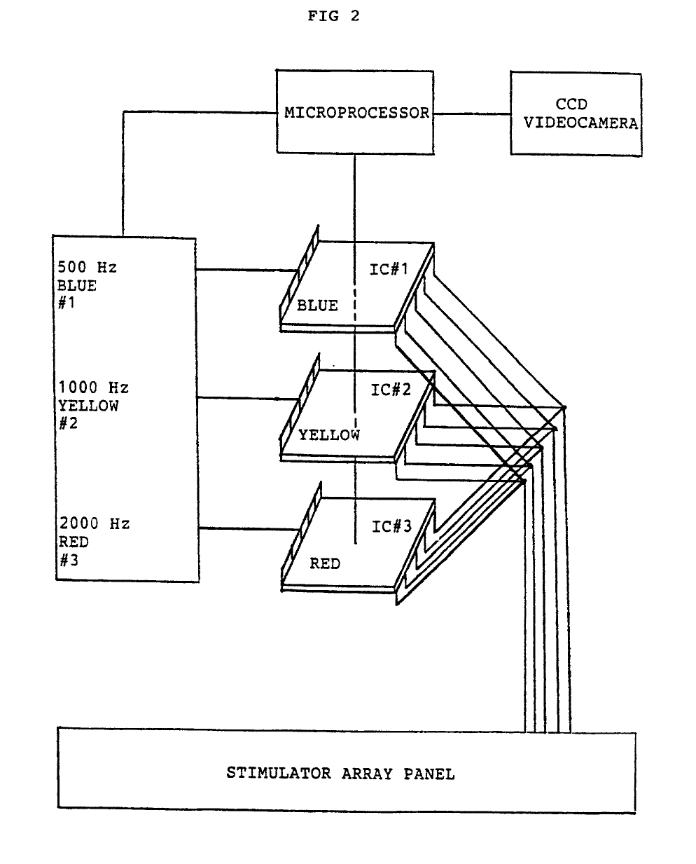

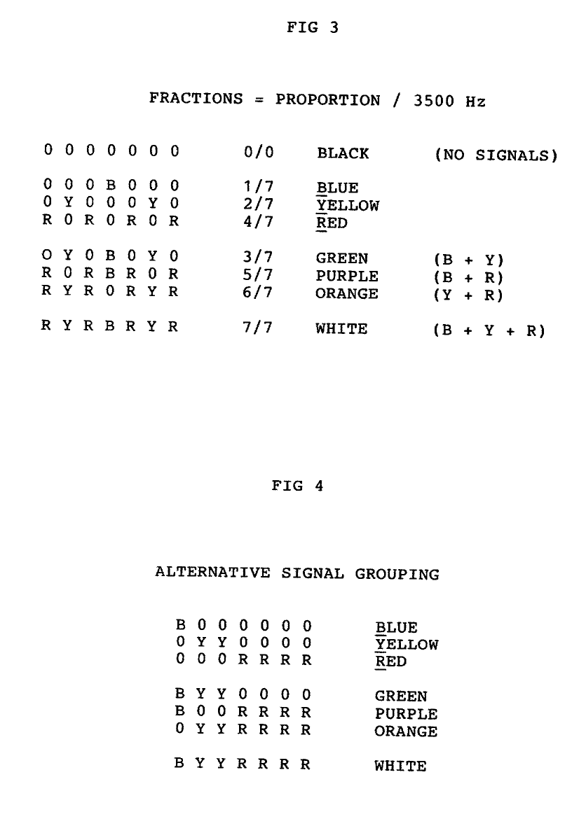

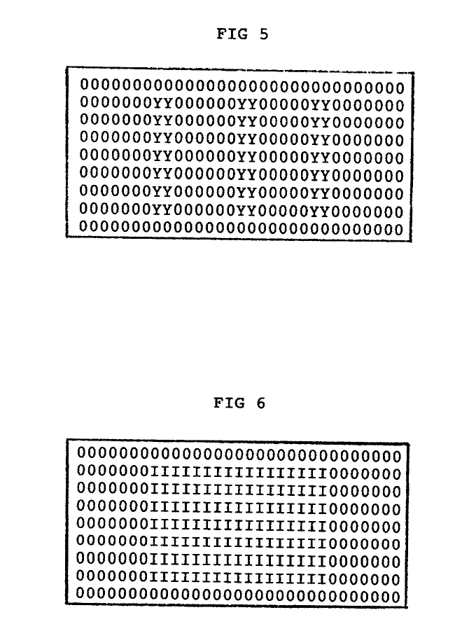

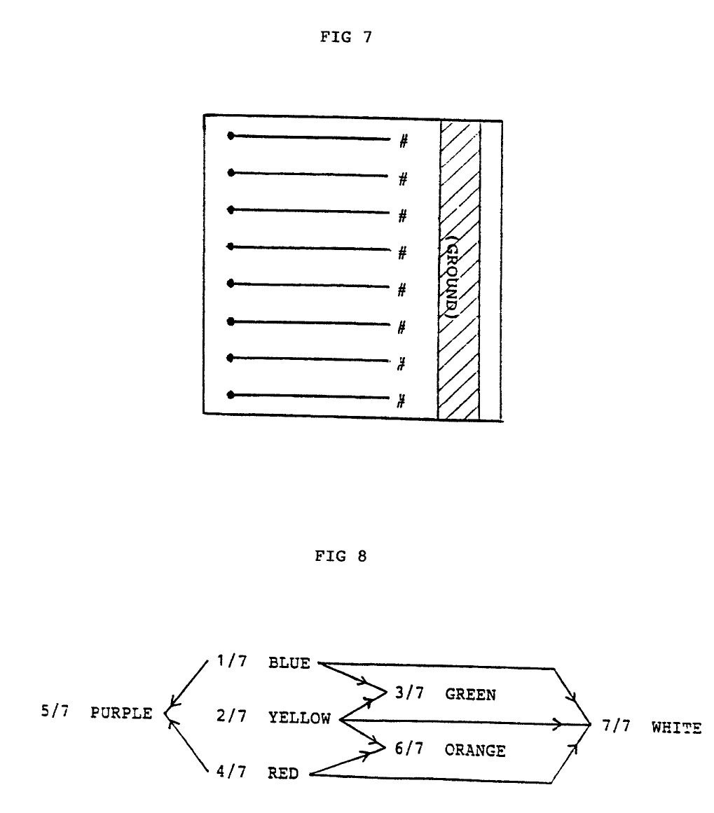

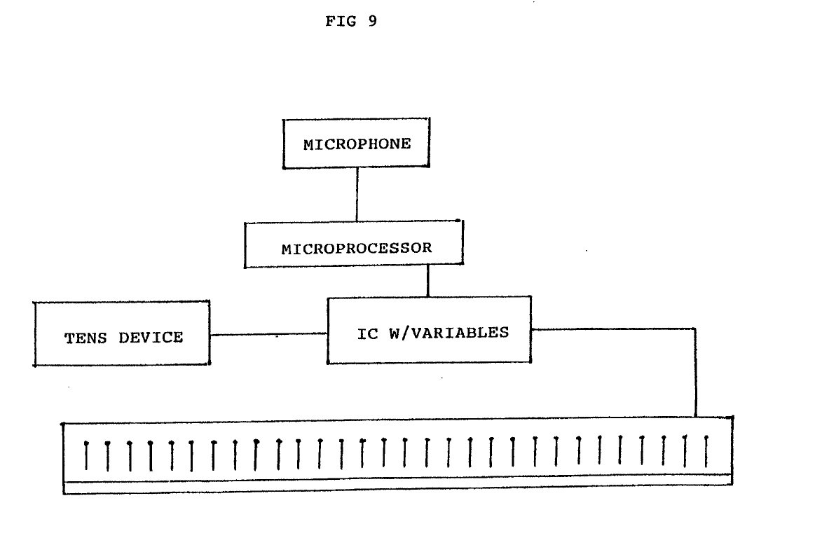

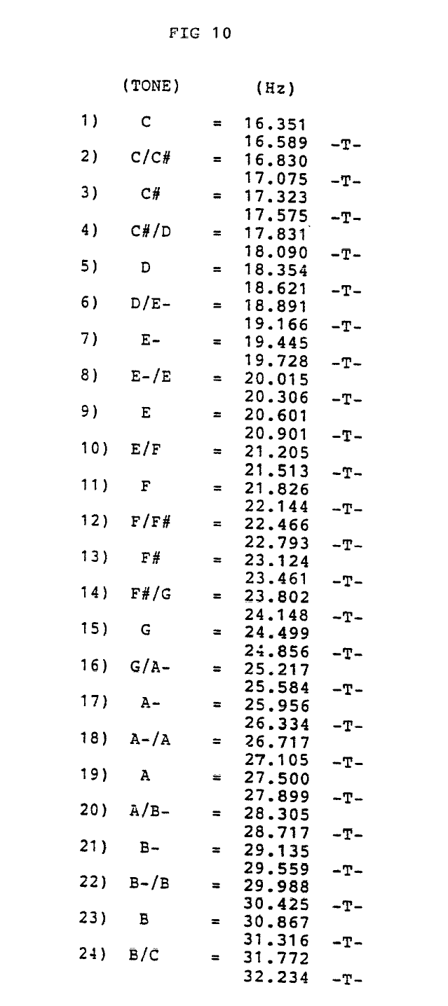

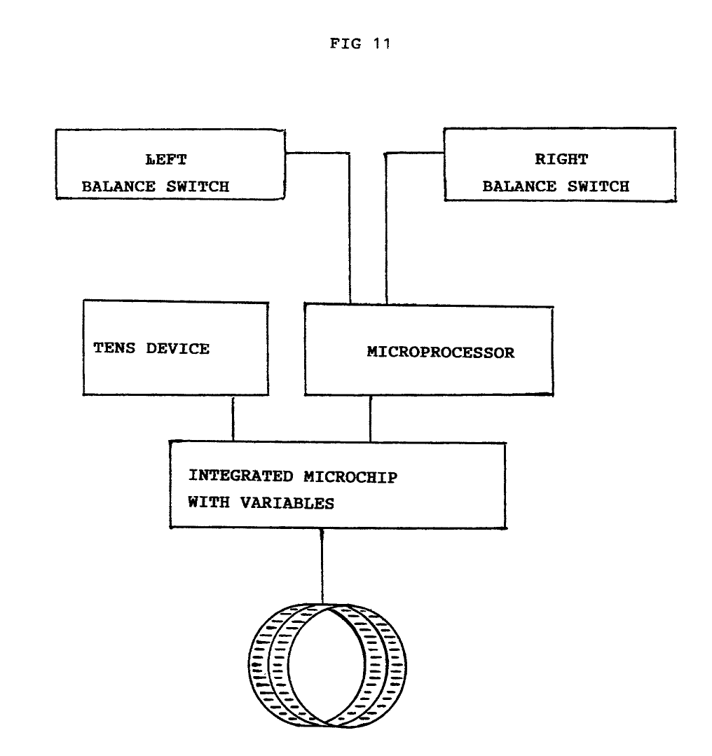

For the Blind, one ‘line’ at a time is delivered to 525 nerve zones, 700 lines at 3500 Hz=FIVE PICTURES A SECOND. Altering frequencies of primary colors yields all hues, and a television quality picture. For the Deaf, electrodes represent sequential musical tones; Each has upper and lower threshold, ‘complimentary’ to neighboring electrodes. Loud tones get more current. Nine octaves signal 216 nerve zones, in an excellent pattern sequence. For Balance Impaired, electrodes circle torso; Tilt, speed of tilt, and rotation signals, operate similarly to balance organs, through two circular arrays of electrodes.

The manner by which SIGHT, HEARING, and BALANCE senses are SYNTHESIZED and fed to hundreds of TOUCH NERVE ZONES, is the content of this invention; But truly it is the characteristics of the SUBCONSCIOUS, that makes it work.

For the BLIND, it will likely take a maximum of five days use, before images begin to form in the visual cortex. Afterward, a television quality color picture is perceived. For the DEAF, understanding of speech will perhaps take several weeks. For the BALANCE IMPAIRED, they will have almost immediate improvement and be well oriented in a few days.

These are bold statements, based on processes of the SUBCONSCIOUS MIND. My documenting publication about those properties was lost, is rare and out of print. I paraphrase quotes from memory, of Futurehealth Incorporated's COMPUTERIZED BIOFEEDBACK, (the Physician's Edition):

“The subconscious is totally un-prejudiced to any signal fed into it, MUST analyze (patterned/organized) signals for any possible meanings, and MUST PRESENT the analyzed-meanings to the CONSCIOUSNESS. It's capacity for deciphering data is great, and has not been measured. The subconscious has NO CHOICE in these matters, but must analyze ALL signals presented to it (if patterned, and having possible meanings)”.

However these Physicians refer to Subliminal (not-audible) Hypnotic therapies, more often harmful than beneficial, these DOCUMENTED parameters reveal a GATEWAY to the consciousness, that may be utilized for SENSE ORGAN SYNTHESIS.

I do not use subliminal audio. I use conscious stimulation on hundreds of nerve zones, by electrodes on a STIMULATOR ARRAY PANEL. These stimulate differentiable “touch sense” nerve groups, with TENS (Transcutaneous Electrical Nerve Stimulation) frequencies. The only important thing yet unknown, is ratios of stimulation zones simultaneously and individually recognized by the subconscious, at highest frequency possible (per number of electrodes).

The OPTICON, invented at Stanford University and Stanford Research Institute in 1971, used a 24 vertical×6 horizontal crystal reed stimulator matrix against the finger pad, enabling the Blind to read PRINTED material at 80 wpm. That limitation exists because stimulators physically vibrate at 250 Hz, and faster reading obscured the letters. My invention uses 525 transcutational electrical stimulators, and operates at 3500 Hz (14 times the frequency of the Opticon), possible because skin is not physically vibrated. Also, I do not “repeat” any signal on a matrix (horizontal) as Stanford did, crediting subconscious to STACK SLICES of a complete video picture every ⅕ of a second (subconscious creating it's OWN “matrix”).

For the BLIND, DEAF, or BALANCE IMPAIRED, the invention differs a little in construction for each. I yield a BRIEF DESCRIPTION of the TECHNICAL ASPECTS, below:

A micro video CAMERA (CCD), or MICROPHONE, or electrolytic BALANCE SWITCHES, serve as inputs; A MICROPROCESSOR modulates the visual, audio, or center of gravity vectors, and commands INTEGRATED MICROCHIPS with VARIABLES. The ICs adjust variables, which act as POTENTIOMETERS, controlling a ‘secondary’ TENS (Transcutational Electrical Nerve Stimulus) potential to pass at varying amplitudes and frequencies, to a STIMULATOR ARRAY PANEL having ELECTRODES 2.5 cm in length and 1.2 mm separation, touching the skin (measurements are approximation).

For SIGHT SYNTHESIS: 525 electrodes deliver a vertical SLICE of the picture at “real time”, every 1/3500 of a second. Brighter pixels open variables more, allowing more current to electrodes. Color perception is also achieved, by assigning a SIGNATURE FREQUENCY to each of the three primary colors, which are additive to the white frequency.

For HEARING SYNTHESIS: 216 electrodes deliver a FREQUENCY RANGE of the tones each is responsible for; the separation of the electrodes help with discernment of pitch and volume. The lowest part of the STIMULATOR ARRAY PANEL (electrodes) delivers bass frequencies, sequential electrodes represent ascending tones, with upper frequency thresholds equal to the lower frequency thresholds of electrodes above them (they are ‘complimentary’). Loud tones open variables more, indicating more volume. The IC's command the variables like an audio equalizer (primary circuit), VARIABLES working like transistors, delivering the secondary circuit TENS signals to each ELECTRODE.

For BALANCE SYNTHESIS: Torus shaped multi-pole SWITCHES ⅓ filled with electrolyte liquid, may be worn like behind-the ear hearing aids or built into frames of eyeglasses. These have axes as the semicircular canals of the inner ear, giving all vectors of the head movements at the highest point over the center of gravity. Studying the characteristics of balance orientation, we sometimes turn our heads slightly when losing balance, presumably to orient normal organs best to regain balance; I propose not changing the way God designed the function. Motion, rotation, inertia, center of gravity, all combine to far more complex vectors than may be thought. Each switch has at least 450 electrical contacts, which are “closed” when electrolyte liquid touches them, on the lower third portion of toruses. A MICROCHIP “piggybacked” to these leads, signals MICROPROCESSOR as to acceleration and degree of rotation (angle), microprocessor commands IC with VARIABLES, the IC sets variables to control the secondary TENS potential to ELECTRODES on the STIMULATOR ARRAY PANELS. The (two) panels are circular, worn around the midriff (torso) of the body. A “pulse code” indicates direction and acceleration of all motions, and even rotating ones (as two balance switches are used).

This concludes the BRIEF DESCRIPTION of the TECHNICAL ASPECTS of this invention for the Blind, Deaf, and Balance Impaired.

Stimulator Array Panels (for all Applications):

The STIMULATOR ARRAY PANEL(S) are constructed of PARALLEL WIRE ELECTRODES, each 2.5 cm in length. Three models exist; One has a common ground running perpendicular along side to the individual electrodes, the wire electrodes are of high electrical resistance, with positive contact made at opposite side to the ground. This insures equal voltage is carried to all subdermal nerves, of the individual “zone” stimulated.

The second type has a comb-tooth common ground, interlocking between the individual stimulators. This stimulates nerves between ground and stimulators much closer, voltages do not penetrate so deep, and less power required. But as ground to below and above electrodes may add spacing requirements between stimulators, overall panel length a serious consideration; the panel best suited is to be determined by purpose, battery drain, cost, effectiveness, and panel length. One is chosen for electronic layout diagrams, and others are illustrated in DIAGRAMS SECTION as possibly a better choice.

The third type has no “common ground”; Sequential electrodes may be “opposite polarity” (+<−/+<−) “paired”, (or +<−>+), by a return circuit to the ICs. Every other pair might be charged, next signal charges formerly omitted ones. This third type is a relatively new concept to me, but I will try to apply it to any application in last edit, when parameters permit it. Some adaptation of this concept is expected to be favored.

The LENGTH of the wire electrodes (2.5 cm), insures a GROUP of touch nerves are stimulated, so that DIFFERENTIATION of “zones” is quickly discerned by the subconscious. This is important, as it greatly reduces spacing requirements between electrodes, and length of the STIMULATOR ARRAY PANEL.

SPACING requirements may be adjusted, only experimentation will yield the best distance between parallel wire electrodes. For design purposes in this application, mine are set at 1.2 mm apart. CONSCIOUS ability to discern one electrode from another, is NOT the means to finally determine best separation. The SUBCONSCIOUS might discern between closer electrodes, than the consciousness is capable of doing. An ORIENTATION PROGRAM in microprocessor operates electrodes in sequence, as “training” to subconscious of sequence and separation of zones stimulated. This may be operated on command or done automatically (a regimen to be established, an unknown at this time).

Length of STIMULATOR ARRAY PANEL required for individuals, will vary. Children have more nerve zones, and may use much shorter panel than required by adults. Generally, the length required (by separation of electrodes) is determined by height.

Wire harnesses containing many wires as presently used inside computers, are very prone to breakdown with the movement they will encounter (linking ARRAY PANEL with the MICROPROCESSOR). The shorter the harness, the better. This means MICROPROCESSOR should be near to the ARRAY PANEL. “Hard wiring” panel to it is impractical, the panel and/or wire harness will need replacing often enough, and one buss connector will attach panel to the microprocessor/TENS source/battery (one enclosure).

MICROPROCESSOR will be worn on a band around the neck, to shorten distance of wire harnesses, to protect them. Heavier, longer lasting battery packs may be worn on belt or where convenient.

A nylon or spandex “body net” may be used to hold the ARRAY PANEL to the body. Velcro will hold the panel securely to the body net. I suspect this would be the easiest way to attach early prototypes, maybe favored entirely. The body net should be a “wrap around” (not a “slip on”), and fastened in the front easily by the user, so as not to damage electrodes.

A future nerve stimulation method, hopefully near as technologies I am unfamiliar with are used, will be subdermal “implants”, activated by a “smart skin” worn over the area. Coils woven into fabric of smart skin induces current in electrodes. The method of implanting electrodes may need to be invented. A tatoo machine, rolled over area to be “impregnated” with electrodes, “shoots” tiny pellets, wires, carbon filaments, invisible electrostatic or conductive ink, (whatever best), to desired subdermal depth of skin. The “smart skin” would find electrodes, microprocessor puts nerve zones in sequence, and “activates” implants. A great improvement if outer hardware is lighter, simpler, cheaper, microprocessor harness less prone to damage. It could be much simpler than it sounds, leaving STIMULATOR ARRAY PANEL (used at present) obsolete.

INTEGRATED MICROCHIPS with VARIABLES (ICs) make this invention possible, without costly and complex electronics. A PRIMARY signal “sets” all the variables constantly, at UHF speed. A SECONDARY higher voltage common to all variables' inputs, is thus controlled individually to the stimulator electrodes.

PROGRAMMING of MICROPROCESSOR(S), parameters of operation, designs of STIMULATOR ARRAY PANELS, are described throughout text of this document. Descriptions accompanying DIAGRAMS SECTION make reference to SECTION NAME and PAGE NUMBER, of written material. Many parameters must be adjustable in early prototypes, since there are unknowns needing research, before final model can be produced with “set” frequencies, voltages, thresholds, programming, electrode spacing, etc.

Sight Synthesis

The MICROPROCESSOR (for vision synthesis) captures the real time video picture from a ‘Charged Coupled Device’ (CCD) micro camera, and splits it into 700 VERTICAL SLICES of 525 pixels each. Normal television gives 525 HORIZONTAL electron beam scans, but microprocessor delivers the picture as 700 vertical slices, sequentially delivered every 1/3500 second.

Also, the COLORS are divided into the PRIMARIES (red, yellow, and blue), not as TV broadcasts. TV stations transmit red, yellow, and GREEN; adding extra signals in the blue frequency (that are removed afterward). After microprocessor captures the picture, the TRUE primaries are divided/modulated and sent to THREE ICs (one each for each of the primaries).

Three different TENS frequencies, are supplied to common inputs of three IC's variables; 500 hertz for blue, 1000 hertz (offset intermittent) for yellow, and 2000 hertz (offset intermittent) for red. Combined frequencies make 3500 Hz, indicating “white”.

The physicist may say, “But blue is more energetic than red, it should have higher frequency”. The conic light receptors of the retina are more vigorously stimulated by RED light, being that it is bent less and more of it hits nearer to the nerve; Lens, prism, and interferometer evidence, proves how the eye is fooled. Color perception is not the mystery claimed. Normal sight DOES perceive red, as if more “lively” than blue.

BLUE=1/7, YELLOW=2/7, and RED=4/7 (of 3500 Hz). If the frequency spacing is perfectly even, they will NOT intermesh synchronized to form the myriad of hues desired, so I have assigned OFFSET INTERMITTENT frequencies for yellow and red, allowing synchronization with each of the others, to form ALL combinational hues and saturations of the primary colors.

Notice the fractions, which divide the total frequency of the Stimulator array panel, if added together=One. The “white frequency” is divided into primaries, that adding any two or three yields independently discernible ones, not confused with any other combination. 1/7, 2/7 and 4/7, a geometric progression with common ratio=2, a finite set whereas added=1, does this. To clarify; Combined primaries form purple (5/7), green (3/7), orange (6/7), and white (7/7) SIGNATURE FREQUENCIES.

A chart of how the primary colors' SIGNATURE FREQUENCIES will combine to form individually discernible ones, is found in the section with the diagrams.

I own an advanced model TENS device, and personally certify even the offset intermittent frequencies and combinations used, are individually discernible from all other combinations. They will stand up to scrutiny.

Slower delivery of complete pictures than ⅕th second delay between them, could adversely affect the Blind enough to cause accidents. ⅕ second delay is long, compared to normal sight. Users could not operate a motor vehicle, unless other normal “reaction times” are unusually fast. Still, as the SLICES of the picture are delivered ‘real time’ to the visual cortex, SOME ‘reaction time’ events will be surprising. When a picture is being completed on the right side, ⅕ of a second will have passed since delivery of the LEFT side of the picture. If you threw a ball from the left side that moment, they might not see it until AFTER it hit them.

The MICRO CAMERA (CCD) is mounted on the bridge of sunglasses. A model already exists for surveillance purposes, a wire runs down behind ear to video recorder on security officer's belt. For my purpose, wire goes to MICROPROCESSOR instead.

To synthesize even CENTER OF VISION, a specialized wide-angle FISH EYE lens, blows-up the center of the picture larger than peripheral vision. Thin replacable plastic loops extend from the glasses' frames, touching the face where muscles can move them, a lever system inside the frames turns camera in any direction without moving the head. The camera turret is tension loaded, the camera returns to forward view when facial muscles relax. The movable center of vision aspect will not likely be perfected in the early prototypes, but advantages will be evident when it arrives; LESS pixels will synthesize BETTER vision, and FASTER pix delivery times made possible.

Sight is NOT organized consciously, of the optic nerve bundle arriving to the visual cortex. A five day old infant can see clearly, not for his conscious intelligence. It is a function of his God given SOUL (subconscious, if preferred), that demands “solution” to the meaning of millions of scattered pinpoints of light. Some organization of those signals is done before birth, because through translucent clothing, bright light does illuminate the interior of the uterus. However, only a few out of focus patterns may be seen by the unborn babe of the umbilical cord, veins and arteries of uterus, knees, and hands. General patterns of light and shadow are established by the unborn. So a person BORN blind, might need more than five days for the soul to first establish some patterns of shapes in light and shadow, before better comprehensive sight is formed.

The vertical SLICES of the picture delivered are pre-organized, so the soul need not do that job, but only to arrange ‘slices’ side by side. It is possible, those NOT born blind will achieve a sharp synthesis of sight faster than my estimated five days, because the invention provides MORE “organization of signals” for the soul; The invention gives BETTER pre-organization of light patterns, than the eye does! However, it is at a DELAY of ⅕ second, as it is inconceivable to deliver ⅓ million signals to ⅓ million nerve zones, for an instantaneous complete picture, at any frequency.

I deliver signals to 525 nerve groups, 700 slices of a complete picture, at 3500 Hz. This equals 1,837,500 pixels per second (almost two million pixels, to deliver five pictures a second).

It is important to realize, the SOUL must be FORCED to create the “pictures” for the mind, and any conscious attempts to decipher the stimulus be THWARTED. Conscious interpretation will NOT synthesize actual sight, but would be like reading braille. This is done, if I am believed or not, by delivering the picture to the STIMULATOR ARRAY PANEL, INVERTED (upside down and backwards, as single-lens normal eye does). This may seem over much ponderance! Research will confirm the necessity of this operating parameter. It will result in UNYIELDING sight synthesis, not “blocked” by other conscious mental activities that could take temporary precedence. It is not probable the consciousness could decipher a moving lens-inverted image, quickly. The subconscious is FORCED to process signals, as it does for normal eyesight. It is entirely POSSIBLE, that CONSCIOUS stimulation of nerve zones is NOT necessary; Actual “subliminal voltages” (not felt), might be all that is needed.

Hysterical Blindness is caused by strong emotional shock, causing the subconscious to cease creating pictures for a time, deeming it's powers assigned to “solving” another problem, more important than even sight. This is strong evidence, that the soul (subconscious, if preferred) has the picture creating ability, and “gates” the images to the visual cortex.

I fashioned an experiment, that deems confirmation by others:

Many of us have done a similar grade school classroom science project, whereby we put a “slit” in a piece of cardboard, and then move the cardboard over the field of vision, while keeping the eye focused straight ahead (not moving). It “writes an image” on the retina that is retained a few moments, if we close our eyes immediately after “scanning” an image.

After conceiving of this invention in 1968 (available electronics made it impractical, then), my question was this; If I MOVED my eye WITH the slit (at a crosshair point drawn midway), could my MIND construct the picture, from only the vertical “slice” painted over the SAME area of my retina? I tried it, and sliding the cardboard over a tabletop (to keep the horizontal crosshair point steady), I SAW a television scene CLEARLY, after only 7 or 8 attempts. My subconscious never forgot how to do this, and after 30 years, a repeat experiment yielded a “picture” in my visual cortex immediately! In 1968 I had no idea the subconscious did this, the result was frightening.

The experiment proves the mind CAN “paint” a complete picture, from only VERTICAL SLICES given to the SAME “slice” of the retina. This does NOT arrive, from a “residual image retained on the retina”. The soul BUILDS a picture, from only vertical “slices” delivered over the SAME points of stimulus, in this one evidential proof.

The experiment also PROVES, that a MATRIX of LEDs delivering a VISUAL vertical signal expanded horizontally, COULD be adapted to the invention for persons with “detatched retina”. This aspect is discussed more fully, in this text.

The best resolution that Stanford's ‘Opticon’ could deliver, with only 24 vertical stimulation reeds at a slow 250 Hz, was 80 words per minute. TENS voltages deliver deeper and sharper stimulation, and does not physically “vibrate” skin surface (far more discernible, and at much higher frequencies). Basic ‘rubber sheet geometry’ shows limitations of the Opticon, due to destructive interference being transferred to neighboring nerve zones through the skin, by physical vibration.

Nerve endings normally for “touch”, are RE-ASSIGNED as the peripheral NERVE CELL CENTER, for other senses. If nerve endings are given clear signal patterns, the subconscious MUST decipher the meanings. Once done, the subconscious never forgets how, and never stops doing it. The soul has “no choice” concerning interpretation of senses' (or any other) stimulus, fully interpreting, and delivering meanings to the CONSCIOUSNESS.

The inventor, myself, has ‘second sight’. It developed in me from hours of working in photography darkrooms. I began to “see” where everything was, as if the lights were ON. If I forgot where something was, my subconscious remembered and let me “see” it. I also practiced and learned the kung-fu ‘Blind man fighting’ technique, whereas blindfolded my subconscious “deciphered” a visual image of weapons swung at me, even if I could not consciously hear the air move, I blocked weapons, and even put sighted assailants on the defense. I learned it quickly, because my darkroom ‘second sight’ was already developed quite well. The actual VISUAL perception I received scared me, and being a Christian I inquired of others about it. They believed demon spirits were giving me this sense of sight when blindfolded, so I stopped practicing the technique. It was not until I learned about COMPUTERIZED BIOFEEDBACK, I realized the subconscious SOUL has these powers (it is not demonic). Now, the manner in which biofeedback has been used, subliminal hypnotic “manipulating” subconscious emotional & reasoning processes, IS proven very dangerous. This invention may be the first totally safe utilization, of the soul's completely unprejudicial and vulnerable ‘gateway’, to the consciousness.

The invention will work so well, the actual VISUAL CORTEX will be “activated” in the formerly blind. To anyone knowing the properties behind it are not “just theoretical”, that conclusion is a given; but few are familiar with backgrounds.

As the ‘movable center of vision’ aspect might not be used in the first prototypes or less expensive models, the below paragraphs must be heeded. For models with ‘center of vision’ hardware, position orientation is not as pronounced a problem; but fine-timing orientation of sudden peripheral movement, will still require a corrective ‘orientation signal’.

In vision synthesis, POSITION of objects in the field of view is easily perceived, most of the time. That is, when extreme left region of picture is different from extreme-right region. But when those slices are identical, as when a few objects are lit in a dark room, the blind cannot perceive WHERE anything truly is in the field of vision, unless an “extra signal” is provided (at borders) to ORIENT the picture.

Also, the 0 to ⅕th second “delay” being an unknown variable, poses problems to reaction timing. If 700 “slices” are delivered constantly, borders and center of vision non-existent, left and right objects would NOT be told apart; if they are there NOW, or WERE there ⅕th second ago. There is a solution:

The PROGRAMMING in the MICROPROCESSOR must include an extra ‘DOTTED-LINE SIGNAL’ on the vertical borders, as frame of reference to “orient” objects, in the field of vision.

IT IS NECESSARY TO DESCRIBE THE NEEDS associated with use of the invention, that the solutions given here have a basis for them. If I simply give operating parameters of the device, it would sound ludicrous to anyone not knowing their purposes. It is an entirely new concept, needing full descriptions of problem areas foreseen, that answers to them are understood.

Synchronization of electrodes “signaling” one color objects is done, that neighboring electrodes do not cause destructive interference (distorting signals) to nearby nerve zones.

As the STIMULATOR ARRAY PANEL is activated every 1/3500 second, HALF of that time is used for “setting” the ICs' VARIABLES, the second half (1/7000 second) of that moment, the variables send the TENS frequency (square wave) to the PANEL.

The MICROPROCESSOR also receives signals from one of the three (internally) synchronized TENS signals, that it may operate the ICs' variables in synchronization to all three frequencies.

From the diagrams, see that the IC's variables' INPUTS are all “hard wired” together (one common), each IC variable receiving full voltage from a TENS device. There are THREE TENS DEVICES, each wired to one of THREE IC's (common) variables inputs. The OUTPUTS of the variables, per pixel on the ‘slice’ represented to the electrodes, the three primary colors (TENS frequencies) are also “hard wired” (pig tailed) together, for each electrode.

It is seen also, that as slices of a picture are delivered at 3500 Hz (one slice every 1/3500 second), a BLUE region will only get stimulation every 7th slice, a YELLOW region will get stimulation 2/7 of all slices, and a RED region every 4/7 of all slices. When combined, all slices get stimulation and indicates WHITE light.

The solution to the dilemma of how to give “fine line” color definition of objects, is to operate the panel in ‘BLACK AND WHITE’ mode ALTERNATELY, with the color perception mode, at the same 3500 Hz. Some pictures are made ‘black and white’ (every color as if white, and every pixel represented).

Remember, none of the primary colors or combination of two of them, activate all electrodes with every ‘slice’ of the picture. For strong “definition”, our normal eyes see only shades of gray in dim light. This being exactly the problem, little line definition of individual primary color objects, BLACK AND WHITE pictures are “staggered” WITH delivery of COLOR pictures. The optimum ratio is an unknown; once determined, the MICROPROCESSOR is programmed for it, that fine-line definition COLOR pictures are “perceived”; This will happen, though only the B&W pix is truly “sharply defined”. Again, the subconscious does this.

3500 Hz reduces complete picture delivery time to ⅕ second (5 pix a second). The frequency should yield good results, as all combinations are exact harmonics to 250 Hz (which was the most discernible skin surface vibration, determined at Stanford University for the Opticon). My method is electric stimulus, not physical vibrations that causes destructive interference through skin tension (basic rubber sheet geometry).

This enhances discernability, and much higher frequencies may be used. The device might however work best at exact HARMONICS of 250 Hz; Every blend of primary colors are, at 3500 Hz; Blue=500 Hz, Yellow=1000 Hz, Red=2000 Hz; all combinations multiples of the base 250 Hz. As the body's electrical nerves operate at 60 Hz, and accidental contact with high voltages safeguarded by that frequency preferred; “touch” nerves are oppositely similar, most DISCERNIBLY stimulated at 250 Hz (physical vibration, not transcutaneous electrical).

Harmonics to 60 Hz may be tried, as frequencies and operating parameters are flexible.

In first prototypes, frequency must be adjustable. TWO and a HALF pix per second (for example), is 1750 Hz (B=250 Hz, Y=500 Hz, and R=1000 Hz). The number of STIMULATORS, or number of ‘slices’ per complete pix may be reduced, making faster pix per sec. possible. We cannot know what is optimal, before prototypes yield this information. An array panel consisting of 60 vertical pixels and electrodes delivering a picture of 80 slices, if found to be the maximum the nervous system could deliver to visual cortex efficiently, it would still be a raging qualified success by any present standards.

The overall amplitude range is adjustable by THRESHOLD CONTROL. When the skin is perspiring or gets salty between washings, skin resistance to electricity is reduced. This would pose problems of uncomfortable or painful voltages, which work fine when skin is dry and clean.

The LOW and HIGH THRESHOLD (to be consciously felt), will need to be “set” for EACH primary color's output signal. This adjusts primary colors' amplitude, for best discernment of all hues.

As “two-wire transmission/reception” of television signals is engineered for other purposes, it would be good to incorporate the technology for my device. Thin coax cable may be damaged too easily, disrupting picture signals to the microprocessor being more than “a minor inconvenience” to the Blind.

The ARRAY PANEL for the Blind is 63 cm long (slightly over two feet). 525 electrodes at 1.2 mm separation, yields a panel length somewhat cumbersome, though no wider than adhesive tape. It will reach from the neck to mid-calf on most adults, going over a hip and down one leg. If electrodes' separation optimum is closer, panel may be made shorter.

STEREO VISION is possible, WITHOUT using TWO Stimulator Array Panels; Two CCD micro cams on sunglasses, signals from each are delivered consecutively to the SAME panel. Two Stimulator Panels may be used, but ONE panel will actually perform BETTER than two, since separation of signals is more quickly discerned.

If delivery speed of slices creates an “astigmatic effect”, LOWER the frequency, use more SEPARATION between electrodes, use LESS electrodes. 60 linear×80 ‘slices’ per complete pix at 1000 Hz yielding best results, is far below expectations. I am confident the subconscious can handle 525, not just 60 pixels, and at the higher 3500 Hz; We must verify optimums.

For people with DETACHED RETINA, having some vision remaining, a MATRIX PANEL on the inside of sunglasses lenses may REPLACE Stimulator Array Panel. FIBER OPTICS can carry the 525 linear (vertical) signals (from LCDs) to illuminate horizontal FRESNEL BARS. VERTICAL signals are given entire MATRIX, sequentially. How fast can it be operated? Perhaps much faster than STIMULATOR ARRAY PANEL (limited by touch nerves' frequency discernibility). Specialized fresnel optics made linear (straight line etch), can bring 2-D close up focus on the matrix for line definition, no other focal axis is necessary. I've met people with detached retina, laser re-attachment has limited success, heralded falsely as “a cure” for the malady. My previous descriptions of how a linear slice repeated on the SAME AREA of the retina, proven utilized by the SUBCONSCIOUS to build a picture, is expanded over MORE retinal area. My subconscious works no different than yours or theirs. The experiment described earlier proves it WILL work for these people, though we do not know “how many pix per second” is possible. ‘Recovery time’ for the conic light receptors of the retina, SEEMS to be less than 50 per second. This is television pix delivery time, that makes TV pictures “flow together” as a smooth moving picture. Conic receptors might have much faster TRUE ‘recovery time’, at least to discern “sequential” signals to the brain; this needs to be learned. If 50 signals per second is the maximum speed that can be utilized, the fiber optic fresnel panel (as designed) could only deliver 1 and 1/14 pix every three seconds. This is extremely slow, expecting much faster delivery is possible. Yet, a more “centralized” zone of vision or less resolution (less ‘slices’ per pix) will increase pix delivery times, to acceptable tolerances (of delay). Modifications of horizontal fresnel shape may be made, for localized areas of damaged retina.

It will not take particular rationale or research, to confirm the above modification WILL work. If initial doubt exists about the other applications, it is expected. My experiment of 1968 described earlier being confirmed, is sufficient proof for at least the above utilization.

Returning to the transcutaneous method, below:

Microprocessor will command overall amplitude of TENS signal with EVERY ‘slice’ delivery, by means of a ‘MAINS VARIABLE’. If THREE electrodes are activated on one slice, amplitude at ‘common input’ from TENS devices is reduced proportionately (3/525th). Proportioned decreases in amplitude calculated by microprocessor per less ELECTRODES stimulated, is a PROGRAMMING parameter. Simpler ICs are used, as 525 variables' tolerance capacity is lowered by a ‘MAINS VARIABLE’, at the junction of the secondary circuit TENS signals (incorporated at the ‘common input’ of the variables).

One television model has 33 increments of saturation, for each of the three primary colors. Whereas range is adjusted with number of pixels activated per ‘slice signal’, increment widths change with it. In short, ICs receive signal to adjust MAINS VARIABLE, and re-incrementation of all other variables with each slice, is not desireable or necessary.

A little imagination will help understanding, of how the entire ‘Sight Synthsis’ system presents a moving picture to the Blind. The Blind can know your “appearance”, by allowing them to run their fingers over your face. Art is made into “raised relief” editions, so they may “see” paintings; by the sense of touch. Imagine instead of their fingers, a strip of SKIN on their backs is “moved across a raised relief picture”, over and over again; The “picture” is the everyday world in front of them, all colors are even discernable, as the “vibrations” (frequencies) change with hues (combination of primaries), and saturations (brightness of any primary, indicated by amplitude).

If you have any imagination at all, you know what this invention means to the Blind, by comparing above illustration.

Hearing Synthesis

For the DEAF, MICROPHONE is input to MICROPROCESSOR, which acts as 216 band audio-equalizer. This signals an IC with 216 VARIABLES, which control a secondary circuit similarly to sound wave cycles, to the STIMULATOR ARRAY PANEL.

The SECONDARY voltage in this case, is straight DC current, the FREQUENCIES are entirely controlled by the VARIABLES.

Each electrode receives only one “tone range” (if a tone is represented). Normal hearing is 16 Hz to 8000 Hz, containing nine octaves of 12 notes each (if divided musically).

I multiply the notes two times per scale, using the forty-eighth root of two, to yield the tones and put them within individual frequency ranges. This gives 216 tones instead of the normal 108 true musical notes, in the nine octaves' range. The math will actually give you 432 tones, the extras are ‘thresholds’ that are ‘complimentary’ between electrodes frequency ranges.

Each electrode passes a sine wave voltage at the same frequency of the tone range; frequency may change within upper and lower thresholds, or encompass as many electrodes as necessary.

The sine wave is DIRECT CURRENT. The effect is more easily grasped, if diagrammed as a continuous sine wave. Half of the full wave (unlike sound) is low amplitude. Sound actually has opposite but EQUAL amplitude, with every half-cycle.

MICROPROCESSOR “splits” and REMOVES every half wave, of the full sine wave, a “square tooth” formed at zero amplitude (where the low amplitude half-cycle wave was).

Sound waves are alternate pressure/vacuum ripples in air, the “vacuum” half cycle” LOWERS current through microphone, the “pressure half cycle” RAISES current. It is the NUMBER of cycles that equals frequency (not half-cycles). The full wave delivered to electrodes does NOT characterize true audio perception (unless they are altered).

Speaker cones pressurize and depressurize air, exactly like sound acts on the microphone diaphragm. Pressurizing and then RELAXING nerve zones for the other half-cycle entirely (no current), does not “mimic” sound waves exactly, but FREQUENCY is preserved (cycles per second, but signal duration halved).

I am wary of CONSTANT electrical stimulus to nerve zones with NO REST to them, from exposure to some sounds. There is no evidence it would damage nerves, but touch nerves can be overworked and “desensitized”. An intermittent depleted frequency (as above does) enhances differentiation, and will not “burn out” nerve zones' sensitivity, tempting users to turn up the THRESHOLD CONTROL (lessening sensitivity more). Full synthesis of opposite amplitude half wave cycles, gives zero rest to nerve zones for duration monotones (sight and balance synthesis signals, do not present this problem).

This solution divides full cycles in half, a good idea as 8000 Hz (highest tone represented) is not easily distinguished from neighboring electrodes; 4000 Hz is easier discerned, as there are greater frequency differences between electrodes.

An alternative is to INVERT the low amplitude half wave (giving it representative amplitude), creating two positive waves, with “spike” created at zero amplitude. This would apply if full cycle wave is found to be satisfactory (not likely). Another alternative, is to create one wave of equal duration as the cycle wave (both sides of cycle, as one long wave). Again, this gives zero rest to nerve zones for some sounds.

Alternating current CANNOT be used to represent the cycle wave; It would cause short circuiting through skin, when frequencies signal neighboring electrodes.

Input, processor, IC, TENS, and stimulator panel; are the HARDWARE of my invention. PROGRAMMING depends on operating parameters, many at present are theoretical.

The yielded mode of operation (in section with diagrams), is only a possibility. All operating parameters are flexible and may be changed as needed for programming, frequencies, electrode separation, etc.

To develop a SLIDING SCALE PERCEPTION (as normal hearing does), each electrode has an upper threshold frequency that is equal to the lower threshold frequency of the electrode above it (all electrodes/thresholds are ‘complimentary’). The sensation to the Deaf as the tone rises, is as if a vibration is increased and changes position in smooth gradients, no “jumps” between electrodes; Consciously perceived separation of electrodes is NOT desired. Subconscious separation of electrodes, helps to discern pitch and volume of many sounds at once. Voltage amplitude changes with volume of tones, and “patterns” formed sequentially by sounds and words the normally hearing perceive, are represented perfectly on the STIMULATOR ARRAY PANEL.

Conscious differentiation of nerve zones being not necessary, panel might be made much shorter than my diagram design. The “sliding scale perception” may DEPEND on electrodes being placed closer together. Being important to give sliding tones (varied frequency) that simulates actual hearing, the optimum distance for separation of electrodes is an unknown, to be established.

There are harmonics to most tones heard in everyday life, but we do not audibly hear most of them. If harmonics are several octaves away and seemingly insignificant, all characteristics of sound possible to reproduce, is done. Not all characteristics of normal hearing CAN be reproduced by this device. The range of electrodes' tones, will not divide all harmonics accurately as the normal ear. It is still a close mimic of normal hearing. All tones having ‘timbre’, have a phenomenally smooth and fine signal pattern sequence, with these operating parameters.

VOICE pitch range, is almost always within 500 Hz to 4000 Hz. The achievement of good synthesis of VOICE being most important, a solitary ‘full normal hearing range’ synthesized by itself, might infringe upon extra stimulus (more divisions of tones) possibly needed, for fine comprehension of the VOICE range.

There are solutions, if this is the case:

The notes of the musical scale at equal temperament, have frequencies most easily determined by X/Y coordinates. X=2, and Y=1/12 (the 12th root of 2). Multiply this number against a note, and it yields the next higher note in the 12 note (inclusive) scale. To divide properly into 24 tones per scale, X=2, and Y=1/24. To divide into 48 tones per scale, X=2, and Y=1/48. Voice might be comprehended better if expanded over more stimulator electrodes, sacrificing tones NOT in the voice pitch range, for an array panel of comfortable length taped to the body. Remember to yield twice the number of tones as electrodes, extra tones are thresholds.

Full range of normal hearing is the objective, with good comprehension of speech. TWO panels may be used side by side, one for voice range only, and the second panel for the full normal hearing range. It is known, normal hearing does not differentiate well between the highest tones, or lower tones very well either. We mostly recognize perfect pitch of notes, that are in the voice range (if gifted with “perfect pitch”).

So there are FOUR ways to construct the hearing synthesis mode:

1) For voice only (500 Hz to 4000 Hz), 2) For full range (16 Hz to 8000 Hz), 3) Separate array panels for both, or, 4) ONE panel that may be activated for EITHER 1) or 2), by ‘voice recognition program’ to automatically switch-modes (preserving the full range mode, most of the time).

Other solutions exist, too. The voice when identified by a voice recognition program could be expanded widely, leaving the other tones in normal placements (superimposing, giving precedence to voice), the frequency expansion an artificial enhancement of voice.

Any voice recognition programming must be bypassable. One reason being, that MUSIC might even be enjoyed. A singing voice should not need modification, to comprehend it well.

An audio-compressor in the circuit may be of great help also, in comprehension of voice. Recognize, that full-compression must NOT be adjustable by the deaf. Especially loud sounds are an alert, and a car crashing should not sound like crumpling paper (as it would). A shout has equal volume as a whisper, at full-compression. A yell to “look out!” should not be interpreted to take a casual glance, if an accident is impending. It should be made illegal, to add full-compression adjustability on these devices.

Almost all hearing aids amplify only 500 Hz to 4000 Hz, for the hard-of-hearing. Other sounds when amplified, have been known to distort voice comprehension. This being a feasible problem with full range synthesis, the best means to MODIFY the voice range WITHIN the full range mode, MAY need to be determined and used (though hoped not necessary).

Let no one say that dividing the tone ranges, need not be as normal music divisions. The soul recognizes them and attaches emotional meanings to intonations, especially in voice. There is no mathematical reason known why some tones and blends are pleasingly harmonic, and others dissonant and disturbing. The soul recognizes intonations in voice, interprets meanings to them, and alerts us to things meant in intonations that were not expressed by the actual words spoken. No mathematics over the frequencies has explained it, but separation into regions of full music scales, will best equip the Deaf to interpret the patterns on the STIMULATOR ARRAY PANEL. Audio interpretation of voice is part emotional, based on musical tone divisions changing over a time-line to the subconscious, and my work to retain this aspect must be respected. The subconscious will not make as good an interpretation, of other type divisions of frequencies.

8 MM moving picture systems have maximum audio frequency range of 100 to 4000 Hz, 35 MM movies range is 50 to 8000 Hz, and most hearing aids only amplify 500 to 4000 Hz (voice range). The widest range known audible to the human ear, is 16 cps to 20,000 cps (cycles per second); Possible, if less electrodes are necessary than expected. As frequencies get higher, there is a proportionate increase to number of electrodes required.

These frequency ranges may be of use, if determined that other tone divisions or more electrodes, are necessary. A narrower full range limitation might exist, than expected.

A great many more types of modulation are possible for electric signals, than for actual audio reproduction. This is to the advantage of research to find the best representative patterns, to synthesize voice. Sound can be made fully discernible to the deaf, by proper stimulation of a few hundred nerve zones.

Some people are blind AND deaf (both). Sight AND hearing synthesis may be COMBINED in one device, with TWO stimulator panels. As many of these are very uncooperative with others (not enjoying life much), it will take some doing to keep the equipment worn by them long enough, before the subconscious “deciphers” the stimulus signals. Imagine that time! A body net of nylon, spandex, whatever, can contain the entire system. No tape or adhesives necessary, and until the person realizes the purpose of the CCD camera, it too can be contained in the front of the body net. It would be a ‘temporary model’, be re-usable to another; The proper gear could now be given to the Blind-Deaf, without fear he will want to damage it (once he knows what it is for).

In the same vein, some Deaf are also Balance Impaired, from extensive damage or deformity to the inner ear organs. They may have combined devices, for hearing and balance synthesis. (Balance Synthesis mode, explained in next section).

As sight synthesis was the beginning of my inspiration, it was developed highly before work on hearing synthesis began. Hearing synthesis was more complex than expected. I do not doubt some things are assumed incorrectly. I really prefer another method of delivering ‘frequency range’ to singular nerve regions, more discernible for all tones. Is it necessary to deliver signal frequencies, identical to audio cycles? Perhaps not. ‘Sliding pitch’ is not discerned well at higher frequencies, not even normal hearing discerns slight pitch changes well.

If we test and find the best discerned “wide range” frequencies (high and low), apply the SAME range to ALL electrodes with tone divisions represented ONLY by electrode separation, the discernment of ‘sliding pitch’ might actually be BETTER than normal hearing (for high/low tones). This is an experimental operating parameter, theoretically more realistic. All others are “hopefuls”, highly dependent on conjecture about touch nerves' frequency discernment. This would make ELECTRODES work with patterns that are parallel and exactly harmonic, as with Sight Synthesis.

In the operating systems which have different frequency ranges on individual electrodes, each electrode commanded by ‘equalizer channel’ (variable) of IC, discernment of pitch is still mainly by SEPARATION of electrodes. Pitch discernment changes within each electrodes' range, would depend on nerves' ABILITY to detect small changes of frequency. Some tone ranges would be discerned well; Others, perhaps not well enough. That it be TRIED at my insistence, though perhaps predestined to fail, at least I do not fail to consider the subconscious' unknowns; That would be purely foolish. It MIGHT excel far above all other systems, after the subconscious has time to adjust.

If above system fails to yield discernment of slight tone changes within critical singular electrodes' ranges, an EXPANSION of frequency range will be needed. Since we cannot expand equally through 216 electrodes, use the SAME frequency range for ALL electrodes. Individual tone ranges discerned well, overall pitch discernment yielded by separation of electrodes, all ‘threshold’ tones complimentary-harmonic to high/low end of neighboring electrodes; Using a FULL OCTAVE (of frequency variance) for signature frequency representing any single tone's variations would work, even if it seems somewhat an “overkill”. It would yield excellent sliding pitch perception. “Perfect pitch” would not be yielded, graduating tones partly represented by electrodes' separation and partly by expanded frequency at individual electrodes, but still a good synthesis. I expect subconscious would cause the consciousness to ignore the wide range of frequency on individual electrodes; thresholds are halved or harmonic frequency, and the “jump” between electrodes reduced in sensation. Also, a lower amplitude stimulation would be needed. The user would probably become totally unaware of the wide range of frequencies on individual electrodes, being a ‘signal’ for subconscious deciphering, only the interpretation yielded to the consciousness, not even cognizant of the expanded signals to subconscious. The subconscious delivers only “the message”, never “the code” of the original presentation.

Balance Synthesis

BALANCE SYNTHESIS, for persons with damage to the balance organs of the inner ear, is also accomplished. The balance organs are chiefly the three semicircular canals, of each inner ear. A liquid partly fills them, tiny internal hairs are bent in the direction of any flow, arising from change of the center of gravity. Hairs above the liquid line are not stimulated at all. Nerves connecting the hairs to the brain, deliver the stimulus of the liquid against the hairs. Slight movements stimulate a few NEW nerves, at the moving “liquid line”. The brain takes more notice of any new vector (even with slow movement). We keep our balance quite well, with eyes shut.

The brain has an internal ‘mechanism’ to assess the exact center of gravity from COMBINED vectors. It does not receive signals from normal balance coils, of exact center of gravity. Rather, it judges where it MUST be, from many vectors yielded by the three semicircular canals of each inner ear, and by other senses not even related to balance organs.

If the balance impaired must wear eyeglasses, electrolytic BALANCE SWITCHES are placed in the temple plates of glasses. Glasses must fit SNUGLY, sudden movements would jostle loose ones, and upset proper operation. It is imperative that good operation is maintained, ESPECIALLY during quick movements, when anyone is most likely to lose balance.

If the Balance Impaired does NOT need glasses, a BRACE hidden under the hairline (behind head), extending around and hooking just over the ears, will stabilize the two switches (positioned behind the ears). A clip on the brace clamps hairs close to the skull, so assembly is not jostled easily. If the patient must wear hearing aids, the switches may be contained inside ‘behind the ear’ aids. The ear canal audio stem will eliminate need for any other stabilizing brace (in this model). Another possibility for those who are also hard of hearing; balance switches are miniaturized, telemetered (wirelessly) to the MICROPROCESSOR, when completely “in the ear” aids are used.

Replicating the PLACEMENT of the switches in proximity of the normal balance organs is necessary, because of the way we regain our balance. We tilt our heads when falling or losing balance. This serves to help us orient ourselves (if you've noticed). Also, the ‘center of gravity’ is only straight down, when we are perfectly still. Inertia, momentum, and centrifugal force, all CHANGE the gravity center vector.

The body is flexible, and when moving, the GENERAL center of gravity can change with NO indications to balance organs. Yet the subconscious knows the position and movements of the body, and makes adjustments on it's own. Because of momentum and inertia, judgments are made as to best retain balance, as the subconscious COMBINES information (not from balance organs alone). Sight helps balance orientation when running, and we more easily lose balance in the dark.

The ELECTROLYTIC SWITCHES each consist of three hollow torus shapes, one third filled with electrically conductive liquid that makes contact with 450 or more wires each, completing the circuits. These are many circuits to send through so many hundreds of wires, so switches are “piggybacked” to MICROCHIPS (molded right to the switches). These MICROCHIPS send a signal of any change in position to the MICROPROCESSOR, where signal is processed and sent to the MICROCHIPS with VARIABLES, which control TENS signals to the STIMULATOR ARRAY PANELS.

The ARRAY PANELS yield patterned signals, representing exact center of gravity changes, constantly.

THREE AXES of rotation are measured by each inner ear mechanism, yielding a total of SIX signals. One might assume that only three are really necessary; but a close study of various arc motions possible, shows that we position our heads so that each group of semicircular canals can measure these motions, at different distances (radii) from ‘center of rotation’.

So, the three axes of rotation measured by EACH SET of balance organs, results in TWO SIGNALS constituting TWO centers of gravity. The THREE axes measured by EACH set of semicircular canals, are “combined” in the subconscious.

My device does NOT signal array panels, with two sets of three axes each. This is NOT necessary.

It is COMBINED information that indicates ONLY center of gravity during movement, that is represented on each of the two ARRAY PANELS. This reduces greatly the number of electrodes needed.

The ARRAY PANELS are circular, are worn horizontally around the torso of the body. The foremost portions represent “forward” tilt, backomost “backward” tilt, right side “right” tilt, etc. Radial movements will cause gradiating differences between (two) ARRAY PANELS, preserving “sense” of THOSE motions.

It should be grasped, why it takes THREE axes of rotation to measure center of gravity vector, accurately. Not quite so obvious; It only requires ONE radial axis, to represent this same vector. A flat (horizontal) torus switch could basically only measure direction of tilt, not degrees of it. Yet a fine representation of degrees already measured, may be transposed to “signature patterns” on ONE axis (ARRAY PANEL).

In this respect, the device might be doing part of the job of the subconscious, and therefore more easily interpreted. I can only theorize about it, not knowing what part of the ‘balance sense’ is done by the utricle and saccule, also listed among ‘the balance organs’.

The invention replicates very closely, the operation of the balance organs. The signals are stronger, proportionate to VELOCITY of movement. As sudden movements bend the tiny hairs in the normal balance organs all along the liquid movement area sharply, so too do sudden movements stimulate the panels more vigorously. As velocity and accelleration can be calculated by microprocessor clock (measuring number of contacts every 1/60th second), measurement need not be “sensed” inside switches (as nerves of the balance organs do).

A gyrocompass can be used, though it gives different signals to the microprocessor. Yet, no gyrocompass is inexpensive or small enough to be practical for the infirmed. It would even be better, as sight would not be a necessary added vector to the subconscious (when we run or turn sharply). Even non-impaired people running in the dark (even on a flat surface), or turning quickly, may lose balance because movements sharply alter center of gravity vectors, when sight is not an added “correction” to orientation.

If you close your eyes, and someone brushes the hairs of your arm, you know which direction the motion went, and about how fast. The nerves of the balance organs work like this. The pattern felt at the electrodes as a slow change of angle becomes faster: The side tilted towards gets more amplitude, gaps between signal zones become smaller (toward tilt), and the whole “pattern” moves in direction of tilt, proportionate to speed (measured by microprocessor clock). Or stated another way:

Direction of the electrolyte liquid movement over wire contacts is recognized by the MICROPROCESSOR, a “pulse code” as “blinking arrow” signs of proportionate speed and direction, commands the IC to set VARIABLES like this. TENS signals indicate tilt, velocity of tilt, and even degrees of rotation, to the STIMULATOR ARRAY PANELS, indicating full vectors and accellerations.

PILOTS may “practice” using the device while not flying, then use it with a gyrocompass when flying. Other signals may be superimposed, indicating True North, airspeed, altitude, even longitude/latitude, G-forces, and position of radar objects. This will save many lives a year, now being lost due to pilot error. A pilot ALWAYS knowing (at least) the BANKING POSITION of his plane without looking at instruments, regardless of centrifugal force “fooling” his normal orientation sense in fog or at night, is a BETTER pilot.

Now I say they may “practice” while NOT flying, because one type of ‘touch sense device’ was ALREADY tried, for fighter jet pilots; They did not “interpret meanings” quickly enough, as signals were organized ONLY by the consciousness. The familiarity gained by hours of use on the GROUND, makes the “interpretation of meanings” INSTANTANEOUS. The military model is not portable, operating by hydrolics to inflatable portions of the cockpit back rest.

When inexpensive gyrocompasses for small private planes become available, every pilot should have some hours of required practice with the invention, for maintaining license. Right now, high cost prohibits mandated use in small planes.

The profoundly balance impaired will have dramatic improvement, very quickly. When he leans, the pattern on the STIMULATOR PANEL will instantly move toward the tilt, compressed in that direction, with proportionate speed and strength to the quickness of movement. It will be so orienting, when before they appeared stumbling drunk. They walk into things, fall down steps, trip easily, are injured often, and called clumsy or retarded. Not even listed with the ‘five senses’ in many treatises, it is still ‘the sense of balance’ synthesized by the device.

If a simpler balance switch exists, it will hopefully be utilized. I would hope for one that operates similar to a cursor “joystick”; tracker is a lightweight sphere floating inside a hollow sphere, an oil-film eliminating drag. A magnet weight causes tracker to rotate, weight points to the center of gravity. Outer sphere has “sensors” that locate the three axes magnet position by triangulation for the microprocessor (being the center of gravity). This might indicate center of gravity faster than normal balance organs do. Aside, the many electric contacts (as in other models), is not necessary. If such a switch or similar type can be miniaturized, it should be substituted.

There is no problem foreseen with fabrication of ARRAY PANELS, with the neigboring ELECTRODES put in linear +−+−+− (opposite polarity electrodes, common ‘ground’). It simplifies fabrication and eliminates perpendicular ground. Only ONE TENS frequency is delivered at any one time, electrode voltages gradually change among neighbors, making above design possible. This cannot be done with sight and hearing synthesis, interference would distort signature frequencies and amplitudes.

The BALANCE SWITCHES operate in above manner also, as any other type of ‘contacts’ to electrolytic fluid, would require twice their number (for equal sensitivity).

The THREE torus shaped ‘sensors’ for each BALANCE SWITCH, are aligned as the semicircular canals of the inner ear, (no one axis exactly horizontal or vertical). The angles represented to the MICROPROCESSOR are combined by trigonomic or coordinate functions constantly, always yielding instant and exact ‘center of gravity’ signals to ICs. Virtually no delay of signature patterns delivered to electrodes, will give excellent results.

For Comatose Patients

Comatose patients deemed unlikely to recover, have awakened. The percentage that recovers, have high incidence of relatives that visit them though unconscious, stimulating them with affectionate touches and words. There is something other to appeal to, than the consciousness. These relatives are often Christian, believing the SOUL of the comatose patient hears and receives compassionate words, to “fight their way back”.

One theory, is that the unconscious person would not know the need to “re-connect” with their body and senses, being in a dream state. The relatives alert the subconscious' mechanisms to the problem; Once analyzed, the soul tries “solutions” (and sometimes finds them).

I request using my invention in sight and sound synthesis, upon COMATOSE patients. Even the ones with little or no brainwave activity. “Communication” to the subconscious, should contain compassionately given information concerning his condition, and appeals to “fight” back to consciousness. As psychiatric practitioners know, subliminal hypnotic REDUCES consciousness levels; The OPPOSITE should work. Using television or radio to stimulate a patient, has no therapeutic value. The soul is in a dreamland, and does not know “the need to awaken”.

I cannot design full parameters of operation, I am not qualified. But as God spoke to me about it, I must report that the brain ITSELF may be treated as a damaged ‘sense organ’, and be partly synthesized. The comatose patient needs to “re-connect”, and the subconscious is the only vehicle that CAN connect the physical senses to the consciousness. The sense of touch is more readily recognized subconsciously than the others, and sense organs synthesis operates through “touch”. Certainly, a loving relative would take a patent's hand, stroke his arm, pat his shoulder. Touch, accompanying words spoken, is a strong argument for many recoveries granted “little hope”.

Once the invention is proven in other areas, this one must be given a try. It sounds bizarre, but as God is my Witness, I realized the thoughts about this were not my own, as I could not abandon them. Finally understanding, Jesus to be obeyed, was shown to “not ignore” these thoughts, and ADD this purpose to the others (despite any ridicule it will bring).

Physician's Warning Note:

Though I do not approve of computerized biofeedback therapies, WARNINGS from Physicians about use of subconscious manipulations must be given here. AGGRESSIVE SUBLIMINAL (constant) causes ‘Subconscious Overload’; Stimulus must not drive patient through many or conflicting emotional “states”, must not be highly repetitive. Permanent and severe psychosis will result, if equipment is operated by untrained or abusive technicians. Patient must be monitored closely, if subconscious overload is to be avoided. Case histories show hundreds of patients driven to irretrievable INSANITY, though “treatments” were for mild psychoses. Nurses must NOT be allowed to alter Physicians' programmed regimens. Physicians need CERTIFICATION to use aggressive subliminal technology legally, and no one really knows how to use it safely; Physicians mostly learn how NOT to use it (to be certified).

I would fear most a physician meaning well, leaving the equipment “ON” 24 hours a day. This is not merely dangerous; Professionals warn that this GUARANTEES inducing mental breakdown; “Aggressive Subliminal” (nearly constant), being the greatest offender in biofeedback subliminal therapies. The comatose patient needs to awaken, but with wits INTACT.

### CONCLUSION AND THEOREM HYPOTHESIS

There is NO DANGER indicated for sense organs synthesis on the Blind, Deaf and Balance Impaired. This, even though aggressive stimulation IS rendered to the subconscious, which “spills over” INSTANTLY into the consciousness (within a few days of stimulus). What is warned as a “danger” for psychiatric mind altering practices, called ‘Aggressive Subliminal Overload’, is NOT a danger in SENSE ORGAN synthesis; but an actual BENEFIT to it.

The invention will PROVE THE HYPOTHESIS PRESENTED: Our SENSES are all “GATED THROUGH” the SOUL (subconscious, if preferred). THIS is why subliminal and biofeedback methods are so powerful, as the soul “has no choice” but to analyze all patterned and organized signals, and “deliver the meanings” to the CONSCIOUS mind; I put forth the hypothesis; this is a PRIMARY HIGHER FUNCTION of the soul. God did not “make a mistake” in making the soul totally un-prejudiced and vulnerable to subliminal manipulations; Our natural SENSES “deliver signals” so complex, if the conscious mind excluded all other work but to comprehend them, it could not but “scratch the surface”. Subliminal technologies “feed” things through the SAME doorway, “bypassing” the conscious mind to so strongly affect the consciousness, simply because the subconscious “has no choice” but to do this.

For the “experimenters” playing with COMPUTERIZED BIOFEEDBACK and other technologies to covertly “affect” the thoughts and emotions of others, it is likened to your PREDECESSORS; When electric batteries were invented, men looked for ways to “use” the power. Immediately, they put the sick in tubs of water and SHOCKED them, for “curative” effects. Your “results” are no better! There is evidence that the technology is abused, in looking for ways to use the power. No one can deny that a PROPER “use” of this power, is ‘Sense Organs Synthesis’. Physicians and technical assistants who have used COMPUTERIZED BIOFEEDBACK and similar therapies, will undoubtedly be the FIRST consulted about the “feasibility” of this invention. I quoted substantiating principles THEY know well, but most people do NOT know such things “as fact”. More than enough DOCUMENTATION exists, shadowing any “feasibility theory” (to professionals in the field). Enough of them will agree, it would be IMPOSSIBLE for this invention to “not work”.

It is now a matter of APPLYING documented and proven principles to THE PURPOSE that is the very REASON for them. When prototypes yield initial results, it will amaze many who are familiar with the principles, that no one utilized the MOST LOGICAL PURPOSE for them, before this time. It simply is NOT TRUE that God designed the soul with it's openness and inner workings, for psycotechnic wizards to pattern thoughts and emotions.

I submit the Scripture: “To everything there is a season, and a time to every purpose under heaven”.

Let any God fearing person reason for himself, if the parameters of the soul's proven inner workings are God given, so someone with subliminal sophistries can covertly manipulate another's thoughts and emotions? But these properties of the soul MUST HAVE a proper purpose, as the Bible does not lie.

I contend that God showed me “the purpose” of the properties of the subconscious, WHY the soul has a “no choice” parameter concerning certain type signals, WHY it “must analyze them”, and WHY it “must present the solutions” to the consciousness.

I prayed and ASKED these things; I could NOT BELIEVE God would place a vulnerable “bridge” to our souls, allowing modernized mind control methods to covertly manipulate someone, UNLESS it had “another purpose” that has been PERVERTED.

The soul functions “like an organ”. If God is true, then the “real you” is a SPIRIT (not the soul). The SOUL “bridges” between the SPIRIT and the BODY. Without the soul, the “real you” CANNOT connect to the brain; God gave it that function. HOW it “connects” them, is a mystery ages old; The soul has an “awareness and life” different from the spirit and brain, but also has DUTIES TO PERFORM, that are strongly evidenced.

Theologians claim the soul contains the heart, will, mind, and emotions (that the soul is a ‘container’ for these things). Very little Scripture even remotely suggests such “believism theology”, it is a theology that satisfies questions; “What is the soul?”, and “What does it do?”.

The SOUL studied by man, not just Bible believing man, has been called “the SUBCONSCIOUS”. It evolved into “ways to manipulate” the CONSCIOUS mind. The most startling discoveries have to do with BRAINWASHING and MIND CONTROL. It is documented FACT that BIOFEEDBACK SUBLIMINAL technology can be used covertly to BRAINWASH. Psychiatrists tried it on the mentally ill, but it just sent patients into even deeper psychoses. One thing discovered was that ‘Aggressive Subliminal’ OVERLOADED the subconscious, and the messages intended to gently-filter into the consciousness, began to “spill over” into the consciousness instantly, causing mental breakdown symptoms of Schitzophrenia! They also discovered the soul would NOT IGNORE any subliminal hypnotic (has “no choice”), but “MUST analyze” everything, and “present” the meanings to consciousness. Extremely complex messages at incredible speeds, the subconscious would eventually “solve”, and the consciousness would “become aware” of the full message; This is how researchers know that the subconscious contains over 97% of the mind's total ability and capacity.

The invention delivers constant signals, like ‘Aggressive Subliminal’ does; but there will be no “breakdown” as other mental manipulation techniques cause, when signals “spill over” instantly into the consciousness. This is simply because the “spill over” is a normal consciousness SENSE interpretation, as the soul NATURALLY does for the senses, anyway.

The SOUL does this for the NATURAL senses, without inducing Schitzophrenia. Would it be different for SYNTHESIZED senses? Utilizing the NATURAL PURPOSE of the subconscious, presents no dangers. The SOUL interprets the NATURAL senses for the consciousness, and will interpret SYNTHESIZED “outer periphery” Nerve Centers, because God DESIGNED the soul to AUTONOMICALLY do this job. If the soul's workings can be MIS-used, this is positive proof these workings can be PROPERLY used, especially for the PRIMARY PURPOSE of the soul's HIGHER FUNCTIONS!

The hypothesis is NOT proof for the invention, the INVENTION will prove the HYPOTHESIS; Then the hypothetical statement may be changed from “if” to “so”, and be accepted as Written Proof.

So the invention provides a means to PROVE the Hypothesis (being a popular opinion lacking proofs), allowing Theorem Hypothesis writing to be conclusive, being as the working invention is an end to all argument and debate about it.

This may be accepted as a provable “useful purpose” for the invention, even BEFORE testing it's physical capacities, and thereby legally entitled to Patent Grant (on this basis). This Statement is written positively in early form, but the invention will either prove (invention works) or disprove (does not work) Theorem Hypothesis. Again, knowlegeable Psychiatrists in Computerized Biofeedback/Hypnotic-Subliminal fields, will assure it is truly impossible for the invention to “not work”. It is a shame THEY did not realize this “gateway” used in Psychotherapy, has complete identifying characteristics of normal modulus, for subconscious interpretation of the natural senses; Evidence proving feasibility of such a device, to invent.

Drawn Diagrams Section

For SIGHT SYNTHESIS DRAWINGS (Pages 42-45, following),

REFER to WRITTEN DESCRIPTIONS (Pages 9-20)

For HEARING SYNTHESIS DRAWINGS (Pages 46-47),

REFER to WRITTEN DESCRIPTIONS (Pages 21-28)

For BALANCE SYNTHESIS DRAWING (Page 48),

REFER to WRITTEN DESCRIPTIONS (Pages 29-34)

RELEVANT to ALL THREE synthesized senses,

REFER to WRITTEN DESCRIPTIONS, below:

S.O.S. INTRODUCTION (Pages 3-8)

COMATOSE PATENTS (Pages 35-36)

CONCLUSION AND THEOREM HYPOTHESIS (Pages 37-40)

What I claim my invention is:

1. It is a sense organs synthesizer, delivering signal patterns representative of sight, hearing, and balance senses, through a few hundred touch nerve zones, working because of documented properties of the subconscious mind.

2. It is a tool to prove (or disprove) an ancient hypothesis of unknown origin and of different wordings, which states that the soul (sometimes using the terms subconsciousness or subliminal mind) bridges the gap between sense organs and the seat of consciousness, being an autonomic primary higher function of the subconsciousness, in analyzing complex sense organs' signals, and presenting the pure meanings (not the original signals) to the conscious mind. The greatest proof, is that ‘aggressive stimulation’ leading to ‘subconscious overload’ and the consequent ‘spill over’ into consciousness, will not cause any psychiatric damage (when used for sense organs synthesis); As proven, all other ‘aggressive subliminal’ type therapies have caused acute Schizoid symptoms.

3. It is a medical therapy instrument for use on comatose patients, using the most sensitive and proven effective channel of communication, the sense of touch. It alerts the subconsciousness to the problem (comatose state), which the subconsciousness interprets as “needing a solution”, and autonomically assists in waking the patient.

4. It is an airplane pilot's orientation device, when used similarly to balance organs synthesis, but wired to a gyrocompass instead. When pilots train, subconsciousness learns meanings of patterns on touch nerve zones, which are delivered to pilot's conscious awareness, instantly and constantly; The pilot is perfectly aware of his plane's orientation at all times.
